(19) United States
(12) Patent Application Publication
(10) Pub. No.: US 2021/0338263 A1
(43) Pub. Date: Nov. 4, 2021

(54) SYSTEM AND DEVICE FOR WATERJET NECROSECTOMY

(71) Applicant: Vanderbilt University, Nashville, TN (US)

(72) Inventors: Patrick S. Yachimski, Nashville, TN (US); Federico Campisano, Nashville, TN (US); Pietro Valdastri, Leeds (GB); Keith L. Obstein, Nashville, TN (US); Claire A. Landewee, Nashville, TN (US)

(21) Appl. No.: 17/306,708

(22) Filed: May 3, 2021

Related U.S. Application Data
(60) Provisional application No. 63/018,983, filed on May 1, 2020.

(51) Int. Cl. A61B 17/3203; A61B 17/00

(52) U.S. Cl. CPC A61B 17/3203; A61B 17/00234; A61B 2017/00367; A61B 2017/00323; A61B 2017/0034

(57) ABSTRACT

A waterjet necrosectomy device for providing a controllable waterjet force capable of fragmenting necrosis without damage to healthy tissue. The device includes a housing having an actuator and a connector coupled to the housing. The connector includes a first channel and a second channel. The device also includes a length of tubing coupled to the connector, a nozzle coupled to the tubing, and a wire extending through the housing, the connector, and the tubing, the wire being configured to articulate the nozzle upon movement of the actuator.

### CROSS-REFERENCE TO RELATED APPLICATIONS

This application is a non-provisional of and claims the benefit of U.S. Provisional Application No. 63/018,983, filed on May 1, 2020, the contents of which are incorporated herein by reference.

### BACKGROUND

Acute pancreatitis results in approximately 275,000 hospital admissions and more than $2.5 billion in health care costs annually. Cases of severe pancreatitis with local adverse events, including pancreatic or peripancreatic necrosis, can be associated with significant morbidity and mortality. Over a period of days to weeks after onset of necrotizing pancreatitis, areas of necrosis may evolve to form mature collections of walled-off necrosis (WON) which, when symptomatic, require intervention/drainage.

Endoscopic minimally invasive therapy using a flexible endoscope has emerged as first line, minimally invasive therapy for management of pancreatic necrosis. Current methods for endoscopic debridement of pancreatic necrosis consist of off-label use of devices developed for other indications—i.e., polypectomy snares, biliary extraction baskets, retrieval nets, grasping forceps, etc. There are no commercially available endoscopic devices specifically developed for endoscopic pancreatic necrosectomy. Accordingly, development of an effective device specifically designed and dedicated for endoscopic debridement of pancreatic necrosis would represent a major advance in the field and in patient care.

### SUMMARY

A device for endoscopic debridement of pancreatic necrosis is disclosed herein. The disclosure, more particularly, describes a WAterjet Necrosectomy Device (WAND) for endoscopic debridement of pancreatic necrosis. The WAND includes 1) high-powered irrigation mechanism for tissue debridement, and an 2) ability of device to articulate and direct application of irrigation.

In one embodiment, the disclosure provides a waterjet necrosectomy device including a housing having an actuator and a connector coupled to the housing. The connector includes a first channel and a second channel. The device also includes a length of tubing coupled to the connector, a nozzle coupled to the tubing, and a wire extending through the housing, the connector, and the tubing, the wire being configured to articulate the nozzle upon movement of the actuator.

In another embodiment, the disclosure provides a system for necrosectomy. The system comprises the waterjet necrosectomy device described above, a vessel in fluid communication with a water supply and the second channel of the connector, a valve, and an actuator coupled to the valve and configured to activate the valve to deliver a flow of water from the water supply, through the vessel, the tubing and the nozzle, and into a body cavity to remove necrotic tissue from the cavity.

### BRIEF DESCRIPTION OF THE DRAWINGS

FIG. 1 illustrates an exploded view of a waterjet necrosectomy device according to an embodiment of the present disclosure.

FIG. 2 is a perspective view of a distal end of the waterjet necrosectomy device illustrated in FIG. 1.

FIG. 3 is a top view of the waterjet necrosectomy device illustrated in FIG. 1 and an endoscope.

FIG. 4 is a top view of the waterjet necrosectomy device illustrated in FIG. 1 inserted in an endoscope.

FIG. 5 is a perspective view of a distal end of a portion of the waterjet necrosectomy device illustrated in FIG. 1.

FIG. 6 is a perspective view of a distal end of the waterjet necrosectomy device illustrated in FIG. 1.

FIG. 7 is a perspective view of a distal end of a nozzle of the waterjet necrosectomy device illustrated in FIG. 1.

FIG. 8 is a perspective view of a proximal end of a nozzle of the waterjet necrosectomy device illustrated in FIG. 1.

FIG. 9 illustrates a necrosectomy system incorporating the waterjet necrosectomy device illustrated in FIG. 1.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Endoscopic intervention has emerged as a first-line approach for drainage of symptomatic pancreatic fluid collections (PFC). The initial step in endoscopic drainage consists of EUS-guided access to the collection followed by transmural placement of either a lumen-apposing metal stent or double-pigtail plastic stent(s). The resultant fistula tract allows drainage of PFC contents into the GI lumen.

While most predominantly liquefied PFC achieve complete drainage with placement of a transmural stent alone, PFC containing solid debris indicative of walled off necrosis (WON) often requires further intervention. For patients without complete drainage, a next step often consists of transmural retroperitoneal endoscopy. This can be performed by advancing a flexible endoscope first per os then through the previously created fistula and into the retroperitoneal space. However, in this setting residual pancreatic necrosis may be bulky and/or densely adherent and difficult to dislodge from the retroperitoneal cavity without further fragmentation. Options for fragmentation include chemical or mechanical debridement, the latter of which is facilitated by off-label use of commercially available endoscopic accessories including polypectomy snares, retrieval nets, forceps, and biliary stone extraction baskets.

Limitations to this approach include the following: (1) none of the devices are specifically designed or intended for this use; (2) as such, these devices are inherently limited in their ability to achieve proficient fragmentation of solid necrotic debris; (3) the efficacy and safety of use of these devices for this purpose has not been rigorously investigated; (4) use of multiple devices per case results in equipment waste and excess cost to the health care system; and (5) complete debridement of necrosis is not always achievable in a single endoscopic session, and some patients require multiple endoscopic sessions in order to achieve complete clearance of necrosis. Accordingly, development of innovative technologies dedicated for necrosectomy use, capable of fragmenting necrotic debris while sparing viable tissue, has been identified as a critical need in the endoscopic management of WON.

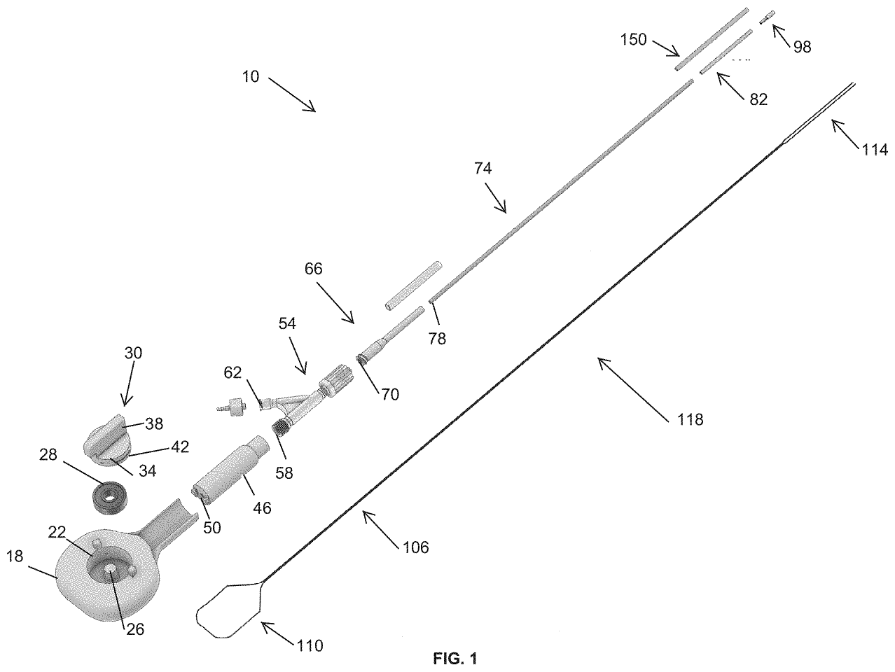

FIG. 1 illustrates a waterjet necrosectomy device 10 for endoscopic debridement of pancreatic necrosis. The waterjet necrosectomy device 10 is configured to provide a controllable waterjet force capable of safely fragmenting necrosis with irrigation alone and without damage to healthy tissue. The waterjet necrosectomy device 10 is a single-use disposable endoscopic waterjet instrument capable of waterjet selection and independent tip articulation.

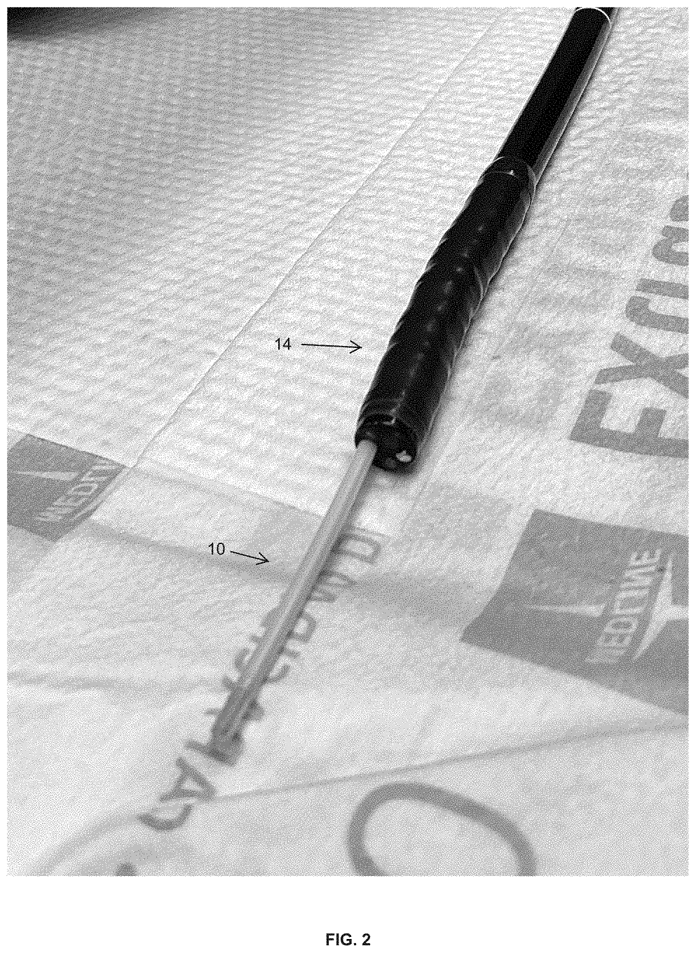

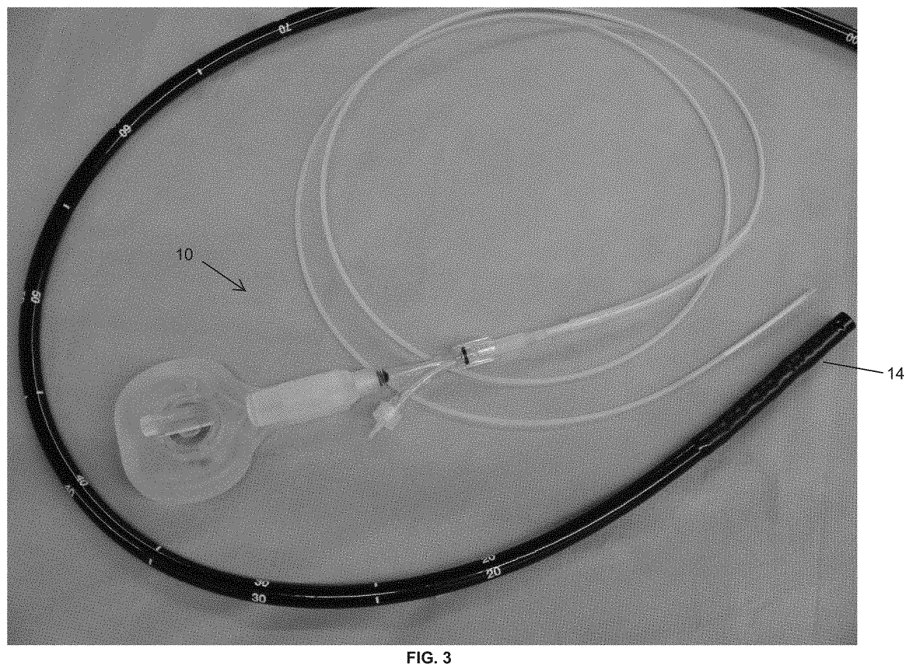

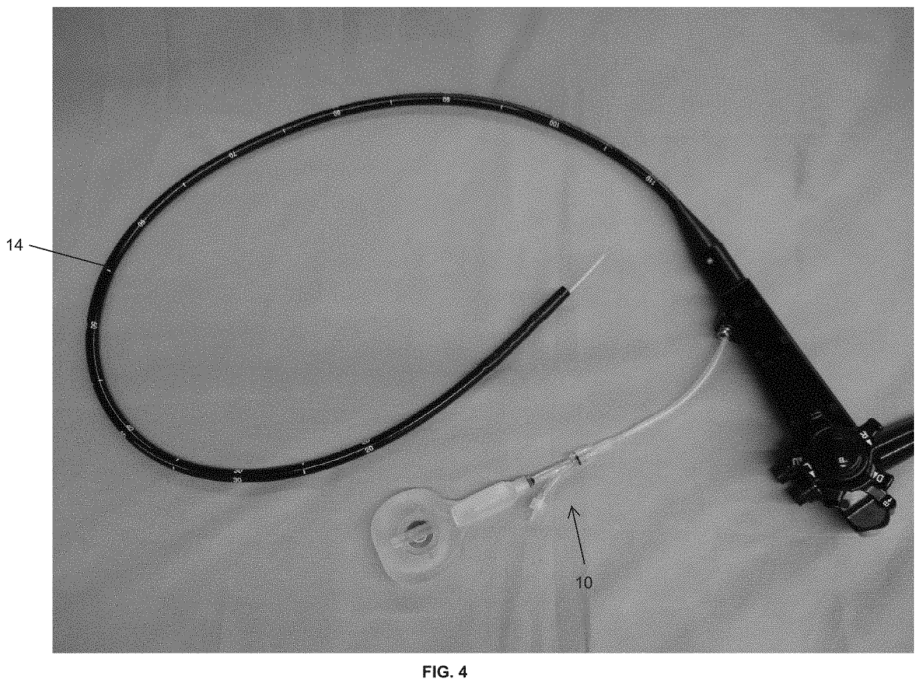

In one embodiment, the waterjet necrosectomy device 10 has a length of about 120 cm to about 150 cm. In other embodiments, the waterjet necrosectomy device 10 has a length of about 130 cm to about 140 cm. In further embodiments, the waterjet necrosectomy device 10 has a length of about 135 cm. In one embodiment, the waterjet necrosectomy device 10 has a diameter of less than about 2.8 mm. In other embodiments, the waterjet necrosectomy device 10 has a diameter of about 2.0 mm to about 2.75 mm. In further embodiments, the waterjet necrosectomy device 10 has a diameter of about 2.66 mm. The diameter of the waterjet necrosectomy device 10 allows it to fit through a 2.8 mm working channel of an endoscope 14 (e.g., a standard adult upper gastrointestinal endoscope) as shown in FIGS. 2-4.

With continued reference to FIG. 1, the waterjet necrosectomy device 10 includes a housing 18 defining a recess 22. The recess 22 includes a post 26 and is configured to receive a bearing 28. The bearing 28 is coupled to an actuator 30 (for example, as illustrated, the actuator is a knob or dial) such that when the actuator is rotated the bearing 28 is configured to rotate about the post 26. The actuator 30 includes a base 34 and a handle 38. The base 34 of the actuator 30 is positioned with the recess 22 and includes a channel 42 on a portion of an outer surface of the base 34.

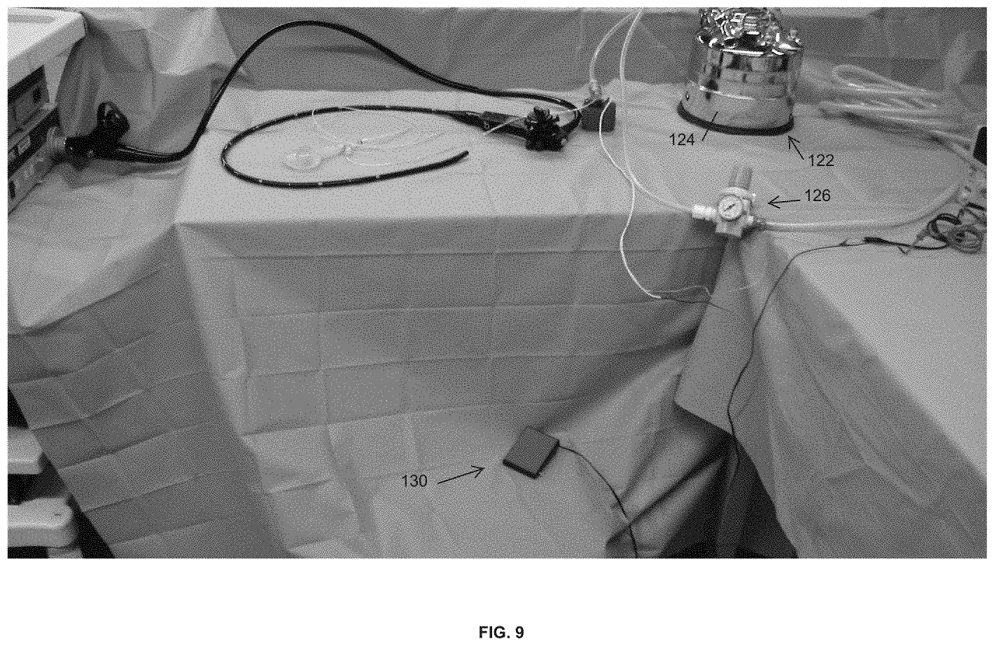

The waterjet necrosectomy device 10 also includes a housing adapter 46 coupled to the housing 18. The adapter 46 includes a channel 50 formed therein. The adapter 46 includes a proximal portion having a first diameter and a distal portion having a second diameter that is less than the first diameter. The waterjet necrosectomy device 10 also includes a Y connector 54 having a proximal portion and a distal portion. The proximal portion of the Y connector 54 is configured to couple to the distal portion of the adapter 46. The Y connector 54 includes a first channel 58 formed therein that is in fluid communication with the channel 50 of the adapter 46. The Y connector 54 includes a second channel 62 that is oriented at an angle relative to the first channel 58. The second channel 62 is configured to connect to a fluid supply 122 (shown in FIG. 9), such as a water supply.

The waterjet necrosectomy device 10 also includes a catheter adapter 66 having a proximal portion and a distal portion. The proximal portion of the adapter 66 is coupled to the distal portion of the Y connector 54. The adapter 66 includes a channel 70 formed therein that is in fluid communication with the channel 50 of the adapter 46 and the first channel 58 of the Y connector 54.

The waterjet necrosectomy device 10 also includes catheter tubing 74 (e.g., biocompatible polytetrafluoroethylene (PTFE)) having a proximal portion and a distal portion. The proximal portion of the tubing 74 is coupled to the distal portion of the catheter adapter 66. The tubing 74 defines a channel 78 formed therein that is in fluid communication with the channel 50 of the adapter 46, the first channel 58 of the Y connector 54, and the channel 70 of the catheter adapter 66.

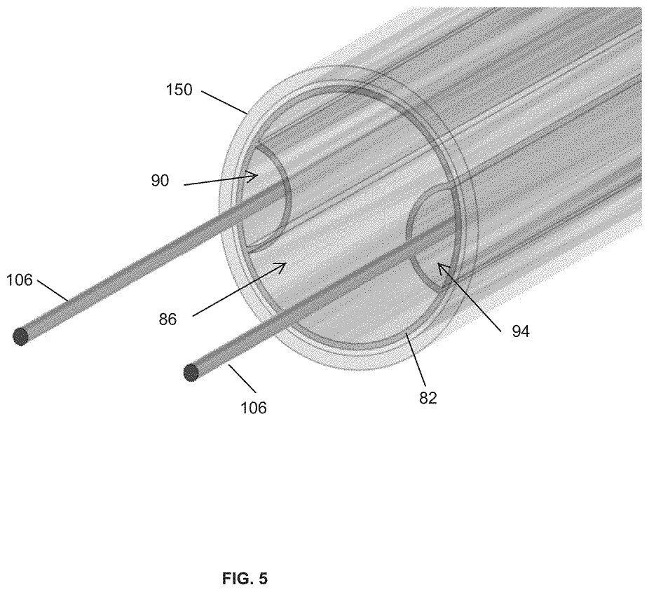

With reference to FIGS. 1 and 5, the waterjet necrosectomy device 10 also includes a wire splitter 82 having a proximal portion and a distal portion. The proximal portion of the wire splitter 82 is coupled to the distal portion of the tubing 74. The wire splitter 82 includes a plurality of channels (first channel 86, second channel 90, and third channel 94). The first channel 86 is in fluid communication with the channel 50 of the adapter 46, the first channel 58 of the Y connector 54, the channel 70 of the catheter adapter 66, and the channel 78 of the tubing 74. The second channel 90 receives one end of the wire 106 while the third channel 94 receives the other end of the wire 106.

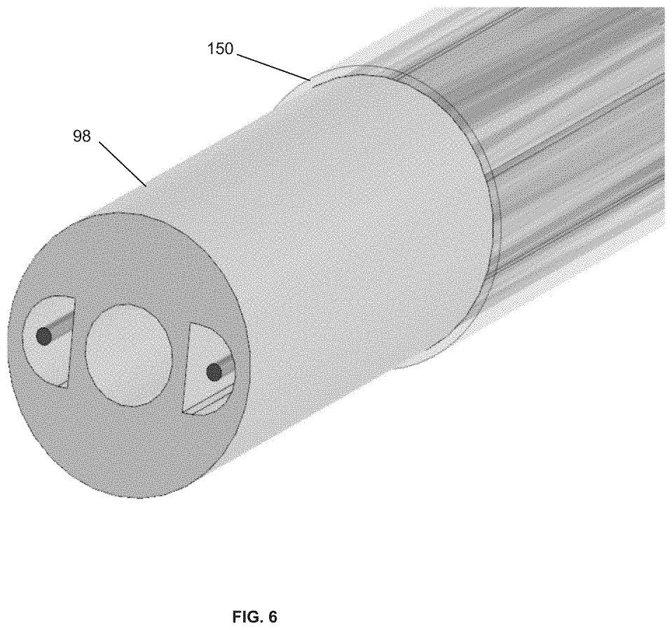

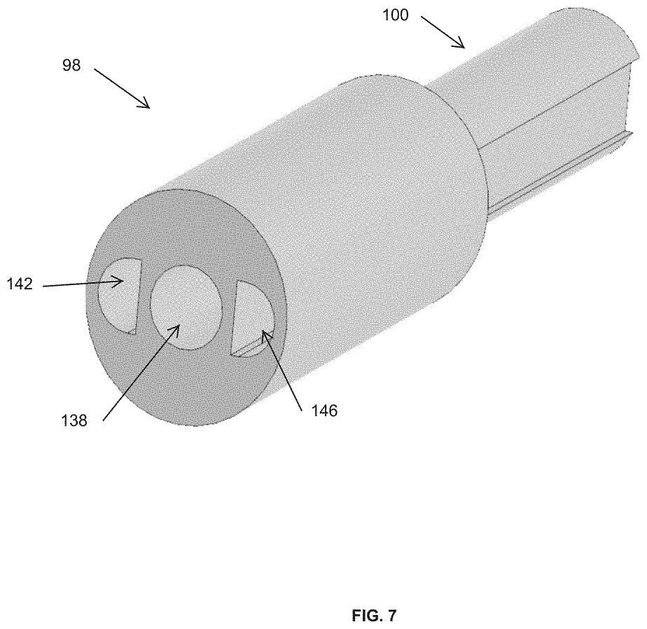

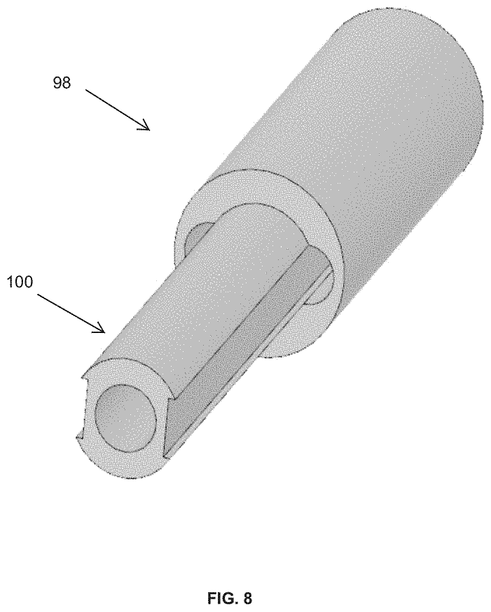

With reference to FIGS. 1 and 6-8, the waterjet necrosectomy device 10 also includes a nozzle 98 having a proximal portion and a distal portion. The proximal portion of the nozzle 98 is coupled to the distal portion of the wire splitter 82. The nozzle 98 includes a portion 100 that fits within the distal portion of the wire splitter 82. The nozzle 98 includes a first channel 138, a second channel 142, and a third channel 146. The first channel 138 is in fluid communication with the first channel 86 of the wire splitter 82. The second channel 142 receives one end of the wire 106 while the third channel 146 receives the other end of the wire 106. The second channel 90 in the wire splitter 82 can be coaxial with the second channel 142 of the nozzle 98. Similarly, the third channel 94 in the wire splitter 82 can be coaxial with the third channel 146 of the nozzle 98.

The waterjet necrosectomy device 10 also includes a bending sleeve 150 (e.g., Pebax tube) which covers the wire splitter 82, a distal portion of the tubing 74 (e.g., about the distal 2.8 cm of the tubing 74), and a proximal portion of the nozzle 98 (e.g., at least the portion of the nozzle 98 that is received within the wire splitter 82). The bending sleeve 150 can be glued, frictionally fit, or the like to the outer surface of the tubing 74 and the wire splitter 82.

The components of the waterjet necrosectomy device 10 can be manufactured using a 3D printing process. For example, the nozzle 98 and the housing 18 can be manufactured using biocompatible photopolymers (e.g., FormLab BioMed Clear or BioMed Amber, FormLab, Mass, USA) in a 3D printing process.

The waterjet necrosectomy device 10 also includes a wire 106. For example, the wire 106 can comprise medical-grade nitinol that does not kink. In one embodiment, the wire 106 is about 0.006″+/−0.003″ gauge. The wire 106 includes a proximal end 110, a distal end 114, and an intermediate section 118 between the proximal end 110 and the distal end 114. The proximal end 110 of the wire 106 is coupled to the actuator 30 via the channel 42. When the actuator 30 rotates on the bearing 28, the rotation is translated to the distal end 114 of the wire 106. The proximal end 110 of the wire 106 is formed as a loop that fits in or around the base 34 of the actuator 30. Just distal of the loop, the two ends of the wire 106 exit the actuator 30 and the housing 18 and the two ends of the wire 106 close and are adjacent to each other. The two ends of the wire 106 remain adjacent throughout the length of the intermediate section 118.

The intermediate section 118 of the wire 106 extends through the channel 50 of the adapter 46, the first channel 58 of the Y connector 54, the channel 70 of the catheter adapter 66 and the channel 78 of the tubing 74. At the distal portion of the tubing 74, the distal end 114 of the wire 106, the ends are separated. One end of the wire 106 extends into the second channel 90 and the third channel 94 of the wire splitter 82. The ends of the wire 106 are separated at the distal end 114 to allow for articulation of the nozzle 98. In one configuration, when the actuator 30 is rotated, the rotation is translated to motion at the ends of the wire 106 to allow for movement of the nozzle 98 over a range of about 120 degrees (−60 degrees to +60 degrees relative to the channel 78 of the tubing 74. For example, when the actuator 30 is turned counter-clockwise, the nozzle 98 moves between about 0 degrees and −60 degrees. Similarly, when the actuator 30 is turned clockwise, the nozzle 98 moves between about 0 degrees and +60 degrees. The nozzle 98 extends from a distal end of the endoscope 14, and therefore, this range of motion is independent of the endoscope 14. This range of motion of the nozzle 98 facilitates precise and accurate targeting of the treatment sites.

With reference to FIG. 9, the Y connector 54 is in fluid communication with a fluid supply 122. More particularly, the second channel 62 of the Y connector 54 is in fluid communication with the fluid supply 122. In one embodiment, the fluid supply 122 is an ASME-Code pressurized liquid dispensing vessel 124 that is regulated (e.g., via regulator 126) to have an entry pressure of 90 psi. This type of vessel 124 has a maximum pressure tolerance of 205 psi at 100 degrees F. In some embodiments, the fluid supply 122 is configured to receive a pressurized gas (e.g., air, CO2) from a pressurized gas source, such as from a wall inlet in a surgical procedure room. The user can then control the pressure in the fluid supply 122 (e.g., the pressurized liquid dispensing vessel) to a pressure at or below 90 psi. The gas compresses water in the vessel and an actuator 130 (e.g., electronic depressible foot pedal) controls a water release valve, giving the endoscopist full control of water irrigation. When pressing the foot pedal, for example, water will flow out of the valve and through tubing to the device 10 with water exiting at the nozzle 98. Irrigation is sustained as long as the actuator 130 is depressed. Irrigation ceases with release of the actuator 130.

The waterjet necrosectomy device 10 can deliver a flow rate up to 0.5 L/min at a maximum surface pressure of 1.3 bar—well below a tissue safety threshold of 3 bar. Both the flow rate and force generated by the device 10 are higher than irrigation volumes and pressures generated by commercially laparoscopic irrigation systems employed during laparoscopic surgery in the peritoneal and retroperitoneal cavities, yet lower than the high pressure water jets currently used for surgical hydrodissection.

### EXAMPLES

Initial benchtop testing used gelatin as a surrogate for pancreatic necrosis. The waterjet necrosectomy device 10 was tested for its ability to fragment different densities of gelatin. The waterjet necrosectomy device 10 was passed through the instrument channel of a gastroscope and was positioned at a distance of 2.5 cm from the gelatin. Irrigation was delivered by the waterjet necrosectomy device 10, both with and without independent articulation of the nozzle 98, with a surface pressure of 1 bar at a flow rate of 0.45 L/min. The waterjet necrosectomy device 10 was further tested on gelatin to confirm articulation and function in a confined environment, by placing the gelatin in a clear stomach phantom. A continuous waterjet force was applied with a surface pressure of 0.72 bar at a flow rate of 0.37 L/min to achieve adequate gelatin fragmentation. The waterjet necrosectomy device 10 was then completely removed from the endoscope and fragmented gelatin was successfully aspirated through the empty working channel of the endoscope. This phase of testing also demonstrated that the waterjet necrosectomy device 10 could be successfully and repeatedly re-introduced through the working channel of the endoscope to deliver further waterjet irrigation without compromising the function of the waterjet necrosectomy device 10. This would allow for multiple cycles of irrigation, fragmentation, and aspiration as would be anticipated in clinical use.

Benchtop testing to assess the device's ability to fragment ex vivo freshly explanted pancreatic necrosis from human subjects. The waterjet necrosectomy device 10 was passed through the instrument channel of a gastroscope and positioned at a distance of 1.5 cm from the necrotic tissue. Irrigation was delivered by the device 10, both with and without independent articulation of the nozzle 98, at a surface pressure of 0.72 bar and a flow rate of 0.37 L/min. This resulted in successful fragmentation of necrotic tissue to remnants less than 2.8 mm in diameter, which is less than the inner diameter of the endoscope's suction channel. Due to success in aspirating gelatin, given its more rigorous and homogeneous composition, aspiration of the brittle necrotic pancreatic tissue after it was fragmented to less than 2.8 mm in diameter was not performed. This phase of testing demonstrated the ability of the waterjet necrosectomy device 10 to fragment pancreatic necrosis as intended for clinical use.

Pre-clinical testing in a swine model. In vivo testing was performed using a 40 kg female Yorkshire Landrace cross swine. This testing was performed in fresh necropsy specimens, within 5 minutes of confirmation of swine death. The goal of this phase was to demonstrate the absence of tissue trauma caused by the waterjet necrosectomy device 10 on non-target, non-necrotic tissue. Effects on the pancreas, small intestine, liver, stomach, spleen, and aorta were assessed. Safety testing for “worst case” scenario was conducted with irrigation at a closer proximity and for a more extended duration than would be anticipated for clinical use. For each target organ, the waterjet necrosectomy device 10 was positioned 0.5 cm from the porcine organ or vessel, and continuous irrigation was applied for 30 seconds over a range of 0.4 bar to 1.3 bar to determine whether any tissue damage would occur. There were five cases of mild tissue blanching and erythema at surface pressures above 0.72 bar. None of the organs or vessels sustained perforation, erosion, or excoriation at any pressures including the maximal pressure for the platform. This phase of testing demonstrated that even when applied directly to nontarget tissue at closer proximity and at more extended duration than would be anticipated in clinical use, the waterjet necrosectomy device 10 creates no significant tissue trauma.

The device 10 is compatible for use with a standard flexible endoscope, and is capable of delivering controlled, targeted irrigation with independent articulation. Irrigation with the device 10 is capable of fragmenting human pancreatic necrosis ex vivo and does not induce trauma to healthy non-target tissue in a swine model. The device 10 offers a novel option for endoscopic pancreatic necrosectomy.

Various features and advantages of the disclosure are set forth in the following claims.

What is claimed is:

1. A waterjet necrosectomy device comprising:
a housing including an actuator;
a connector coupled to the housing, the connector including a first channel and a second channel;
a length of tubing coupled to the connector;
a nozzle coupled to the tubing;
a wire extending through the housing, the connector, and the tubing, the wire configured to articulate the nozzle upon movement of the actuator.

2. The waterjet necrosectomy device of claim 1, wherein the second channel of the connector is coupled to a fluid supply, and wherein the fluid travels through the tubing to the nozzle, and wherein the nozzle is configured to deliver the fluid to a body cavity to remove necrotic tissue from the cavity.

3. The waterjet necrosectomy device of claim 1, wherein the wire includes a loop positioned around the actuator in the housing.

4. The waterjet necrosectomy device of claim 1, further comprising a wire splitter coupled to the tubing and the nozzle.

5. The waterjet necrosectomy device of claim 4, wherein two ends of the wire come together distal of the loop and remain together through the connector and the tubing.

6. The waterjet necrosectomy device of claim 5, wherein the two ends of the wire are separated at a distal end of the tubing.

7. The waterjet necrosectomy device of claim 6, wherein the two ends of the wire extend through a separate channel in the wire splitter.

8. The waterjet necrosectomy device of claim 4, further comprising a sleeve coupled to an outer surface of the tubing, the wire splitter, and the nozzle.

9. The waterjet necrosectomy device of claim 1, wherein the nozzle is configured to rotate about 120 degrees when the actuator is rotated in a clockwise direction and a counter-clockwise direction.

10. The waterjet necrosectomy device of claim 1, wherein the wire comprises nitinol.

11. The waterjet necrosectomy device of claim 1, wherein the fluid supply is pressurized, and wherein the nozzle is configured to deliver the fluid at a flow rate up to 0.5 L/minutes.

12. The waterjet necrosectomy device of claim 11, wherein the flow rate is at a maximum surface pressure of 1.3 bar.

13. The waterjet necrosectomy device of claim 1, wherein the actuator is a knob.

14. A system for necrosectomy comprising:
the device of claim 1;
a vessel in fluid communication with a water supply and the second channel of the connector;
a valve; and
an actuator coupled to the valve and configured to activate the valve to deliver a flow of water from the water supply, through the vessel, the tubing and the nozzle, and into a body cavity to remove necrotic tissue from the cavity.

15. The system of claim 14, wherein the vessel is pressurized, and wherein the nozzle is configured to deliver the water at a flow rate up to 0.5 L/minutes.

16. The system of claim 15, wherein the flow rate is at a maximum surface pressure of 1.3 bar.

17. The system of claim 14, wherein the actuator coupled to the valve is a foot pedal.

18. The system of claim 14, further comprising an endoscope having a working channel configured to receive the tubing of the device, and wherein the nozzle extends from a distal end of the endoscope.

19. The system of claim 18, wherein the nozzle is configured to rotate about 120 degrees when the actuator is rotated in a clockwise direction and a counter-clockwise direction.
